(19) United States
(12) Patent Application Publication
(10) Pub. No.: US 2023/0127401 A1
(43) Pub. Date: Apr. 27, 2023

(54) MACHINE LEARNING SYSTEMS USING ELECTRONIC HEALTH RECORD DATA AND PATIENT-REPORTED OUTCOMES

(71) Applicant: The Trustees of the University of Pennsylvania, Philadelphia, PA (US)

(72) Inventors: Justin Eli Bekelman, Philadelphia, PA (US); Ravi Bharat Parikh, Philadelphia, PA (US); Jinbo Chen, Newtown, PA (US); Jill Schnall Hasler, Conshohocken, PA (US)

(21) Appl. No.: 17/972,105

(22) Filed: Oct. 24, 2022

Related U.S. Application Data
(60) Provisional application No. 63/270,818, filed on Oct. 22, 2021.

(51) Int. Cl. G16H 50/30; G16H 10/60; G06N 5/02

(52) U.S. Cl. CPC G16H 50/30; G16H 10/60; G06N 5/022

(57) ABSTRACT

Methods, systems, and computer readable media for predicting patient outcomes. In some examples, a method includes training, using at least one processor, a predictive model by fitting a first model using patient outcome data for a number of individuals and electronic health record data for individuals. Training the predictive model includes fitting a second model using patient reported outcome data for a subset of the plurality of individuals. The method includes supplying patient data for a patient to the predictive model and using the predictive model to predict at least one patient outcome for the patient.

### GOVERNMENT INTEREST

This invention was made with government support under CA014089, CA197461, HL138306 and CA263541 awarded by the National Institutes of Health. The government has certain rights in the invention.

### PRIORITY CLAIM

This application claims the benefit of U.S. Provisional Patent Application Ser. No. 63/270,818, filed Oct. 22, 2021, the disclosure of which is incorporated herein by reference in its entirety.

### TECHNICAL FIELD

The subject matter described herein relates generally to computer systems for machine learning. More particularly, the subject matter described herein relates to methods and systems for machine learning using electronic health record data and patient-reported outcomes.

### BACKGROUND

Patients with cancer often suffer debilitating symptoms related to their cancer and associated treatment. Patient-reported outcome (PRO) assessment may allow oncology clinicians to better identify patients with high symptom burden or declining functional status. Routine web- or text-based PRO collection is associated with decreased acute care utilization and improved symptom control, patient-clinician communication, health-related quality of life, and even survival. Owing to increasing evidence around the health-promoting benefits of PRO collection, emerging national guideline recommendations, and novel electronic and remote methods of capture, PRO assessment has recently become feasible for large numbers of patients in routine oncology practice.

While PRO collection may improve symptom management, the role of PROs in risk stratification remains unexplored.

### SUMMARY

This document describes methods, systems, and computer readable media for predicting patient outcomes. In some examples, a method includes training, using at least one processor, a predictive model by fitting a first model using patient outcome data for a number of individuals and electronic health record data for individuals. Training the predictive model includes fitting a second model using patient reported outcome data for a subset of the plurality of individuals. The method includes supplying patient data for a patient to the predictive model and using the predictive model to predict at least one patient outcome for the patient.

The subject matter described herein may be implemented in software in combination with hardware and/or firmware. For example, the subject matter described herein may be implemented in software executed by a processor. In one example implementation, the subject matter described herein may be implemented using a computer readable medium having stored thereon computer executable instructions that when executed by the processor of a computer control the computer to perform steps. Example computer readable media suitable for implementing the subject matter described herein include non-transitory devices, such as disk memory devices, chip memory devices, programmable logic devices, and application-specific integrated circuits. In addition, a computer readable medium that implements the subject matter described herein may be located on a single device or computing platform or may be distributed across multiple devices or computing platforms.

### BRIEF DESCRIPTION OF THE DRAWINGS

FIG. 1A is a block diagram of an example computer system for predicting patient outcomes;

FIGS. 1B-1C are charts illustrating univariable and multivariable odds ratios and 95% CIs of association between PROs and mortality;

FIGS. 2A-2D are charts illustrating a comparison between different predictive models;

FIG. 3 is a flow diagram of an example method for predicting patient outcomes;

FIGS. 4A-4B are charts illustrating example data for the usefulness of an example system that leads to cost savings in end of life spending.

### DETAILED DESCRIPTION

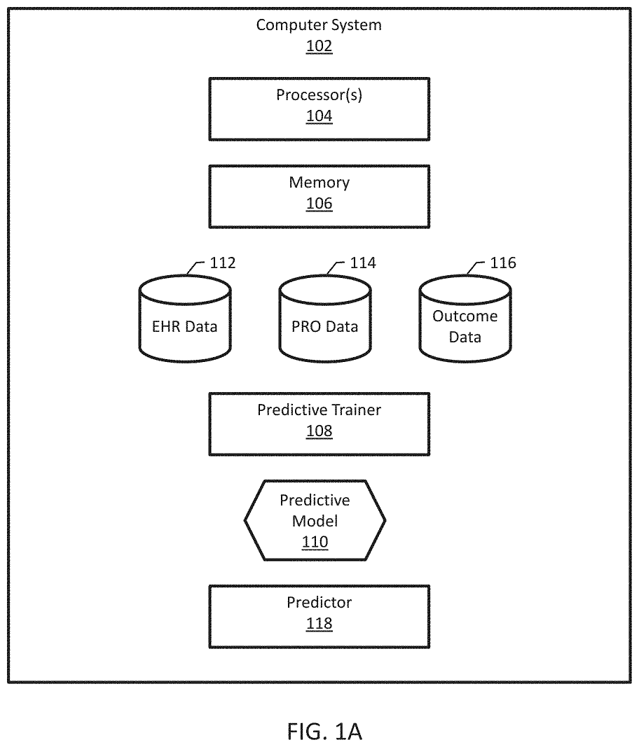

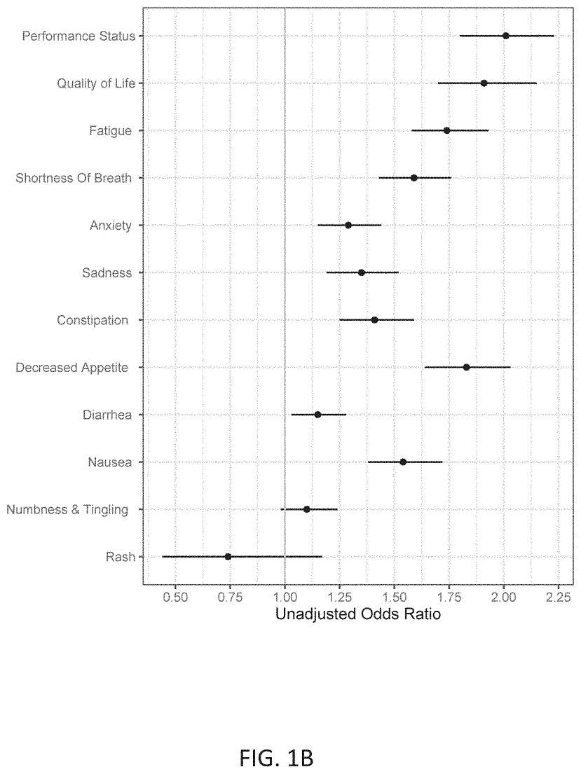

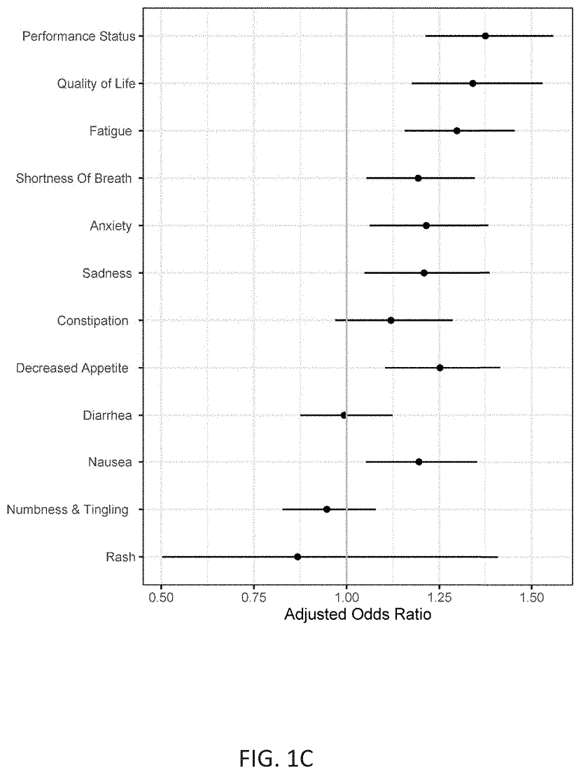

FIG. 1A is a block diagram of an example computer system 102 for predicting patient outcomes. The system 102 includes at least one processor 104 and memory 106 storing instructions for the processor 104. The system 102 includes a predictive trainer 108 configured for training, using one or more machine learning algorithms, a predictive model 110 using electronic health record data 112 for a number of individuals, patient reported outcome data 114 for at least a subset of the individuals, and patient outcome data 116 for the individuals. The system includes a predictor 118 configured for supplying patient data for a patient to the predictive model 110 and using the predictive model 110 to predict a patient outcome for the patient.

The system 102 can be used to predict any appropriate type of patient outcome. For example, the system 102 can be used to predict patient mortality. The system 102 can output the prediction, for example, by displaying the prediction on a display screen to a physician or other appropriate person, or by sending the prediction to a remote computer system.

In some examples, training the predictive model can include a two-phase methodology. Let N denote the number of individuals with phase I data and m denote the number of individuals with phase II data. Denote X the set of EHR variables that we have available for all individuals, Z the PRO variables that we only have available for a subset of individuals, and let Y represent the outcome, 180-day mortality status.

Training the predictive model can include first fitting a preliminary least absolute shrinkage and selection operator (LASSO) model, between Y and X. Using this model, we obtain a summary score for the EHR covariates, Ŷₓ=logit{P(Y=1|X)} and a preliminary predicted probability for 180-day mortality, P¹(Y=1|X). Then, training the predictive model can include fitting a logistic regression model with an offset term,

$$\frac{P ( R = 1 \vert Y = 1 , X )}{P ( R = 1 \vert Y = 0 , X )} .$$

For the missingness models used in the offset term, the smooth terms can be included, f₁{P¹(Y=0|X)} in the model for cases and f₀{P¹(Y=1|X)} in the model for controls, fit using B-spline approximation. In some cases, phase II data is missing completely at random, and any model specified will be valid. Both the preliminary model and the missingness models are fit using phase I data for all subjects even if they do not have phase II data available. Then, training the predictive model can include fitting a logistic regression model with 180-day mortality as the outcome and Ŷₓ and the phase II data as the covariates, and we add the offset term in order to account for the two-phase data structure.

Examples of training a predictive model are described further below with reference to a study performed on example training data.

Although specific examples and features are described in this document, these examples and features are not intended to limit the scope of the present disclosure, even where only a single example is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed in this specification (either explicitly or implicitly), or any generalization of features disclosed, whether or not such features or generalizations mitigate any or all of the problems described in this specification. Accordingly, new claims may be formulated during prosecution of this application (or an application claiming priority to this application) to any such combination of features. In particular, with reference to the appended claims, features from dependent claims may be combined with those of the independent claims and features from respective independent claims may be combined in any appropriate manner and not merely in the specific combinations enumerated in the appended claims.

Introduction

Patients with cancer often suffer debilitating symptoms related to their cancer and associated treatment.^{1, 2} Patient-reported outcome (PRO) assessment may allow oncology clinicians to better identify patients with high symptom burden or declining functional status.^{1, 3} Routine web- or text-based PRO collection is associated with decreased acute care utilization and improved symptom control⁴, patient-clinician communications, health-related quality of life³, and even survival⁶. Owing to increasing evidence around the health-promoting benefits of PRO collection, emerging national guideline recommendations, and novel electronic and remote methods of capture, PRO assessment has recently become feasible for large numbers of patients in routine oncology practice.⁷

While PRO collection may improve symptom management, the role of PROs in risk stratification remains unexplored. This leaves open the question of whether incorporating PROs may improve traditional risk stratification tools used for care management and end-of-life planning. Mortality risk stratification is a potential use case for PRO integration, as oncology clinicians are often unable to identify patients at risk of short-term mortality based on intuition or routine risk stratification tools, instead overestimating life expectancy for up to 70% of their patients.⁸⁻¹¹ Better awareness of short-term mortality risk—usually defined as death within six to twelve months—may inform clinicians' decisions about advance care planning and palliative care referrals and could lead to more goal-concordant cancer care.¹²⁻¹⁷ Recent advances in electronic health record (EHR) infrastructure and machine learning (ML) may identify many patients with cancer at risk of short-term mortality—oftentimes more accurately than clinicians.¹⁸⁻²² However, such ML algorithms usually rely on structured EHR data, including laboratories, demographics, and diagnosis codes, which provide limited insight into patient symptoms or functional status. As a result, ML mortality risk prediction algorithms often have true positive rates under 50%—performance that is suboptimal for clinical implementation.²³ PROs, which have been independently associated with mortality in prior studies²⁴, may augment such ML algorithms.

There is a critical need to understand whether PROs augment ML mortality risk assessment in order to optimize prognostic algorithms in order to better deliver oncologic and palliative care. In this study, we trained and compared 3 ML algorithms based on EHR data alone, PRO data alone, and EHR plus PRO data, to estimate 6-month mortality among patients seen in oncology clinics affiliated with a large academic cancer center. We hypothesized that adverse PROs would be independently associated with 6-month mortality, and that integrating routinely collected PROs into EHR-based ML algorithms would improve predicted performance compared to ML algorithms based on EHR or PRO data alone. By virtue of this approach, our findings provide clinicians and health system leaders with insight regarding the potential impact of PROs on predictive algorithms in oncology.

Methods

Data Source

We derived our cohort from patients receiving care at the Perelman Center for Advanced Medicine at the University of Pennsylvania Health System (UPHS) who were listed in Clarity, an EPIC reporting database, which contains individual electronic medical records for patients containing demographic, comorbidity, and laboratory data. We chose patients in this clinic because 1) there has been routine collection of PROs for nearly all medical oncology patients since mid-2019, and 2) an EHR-based ML algorithm has been previously validated in this cohort²⁵ we used the same data inputs to develop a reference algorithm in this study. Health insurance claims data were not available. Our study followed the transparent reporting of a multivariable prediction model for individual prognosis or diagnosis (TRIPOD) checklist for prediction model development and validation.²⁶ We obtained approval and waiver of informed consent from the University of Pennsylvania institutional review board prior to conducting this study.

Study Population

To develop our model, the cohort consisted of patients aged 18 years or older who had outpatient medical oncology encounters at a large tertiary practice between Jul. 1, 2019 and Jan. 1, 2020. Patients were not required to have received cancer-directed treatment to be included in this study. We excluded patients who had benign hematology or genetics encounters, less than 2 encounters during the study period, or no laboratory or comorbidity data within 6 months of the encounter. The latter two criteria were meant to exclude new patients or patients who did not actively follow with a UPHS oncologist. Our final cohort consisted of 8600 patients. In all statistical analysis and modeling, we used the first hematology/oncology encounter in the study period for each patient as the index encounter for statistical modelling. We chose not to incorporate PRO data from subsequent encounters because we found that trends in PROs were not associated with mortality.

EHR Features

Our EHR data set included three broad classes of features: (1) demographic variables (continuous age, patient-reported gender) at the time of the encounter; (2) 33 Elixhauser comorbidities²⁷ in the entire patient history (total) and 180 days prior to the encounter (recent), which included diagnostic codes for metastatic cancer along with chronic conditions such as congestive heart failure, chronic pulmonary disease, and hypertension; and (3) laboratory data in the 180 days prior to the encounter, including basic laboratories (complete metabolic panel, complete blood count, etc.) and certain tumor-specific laboratories (prostate-specific antigen, carcinoembryonic antigen, etc.). Race and ethnicity were not included as discrete features due to the potential for introducing bias, and performance status was not included due to high missingness. Our strategies for handling missing values and arriving at the final feature set of 559 variables have been previously described.¹⁸ No comorbidity or laboratory data after the index encounter date was included in model predictions.

PRO Features

PROs were assessed using the PRO version of The Common Terminology Criteria for Adverse Events (PRO-CTCAE™)²⁸ and the Patient-Reported Outcomes Measurement Information System (PROM'S©) Global v.1.2²⁹ scales and consists of Likert-scale responses to 12 questions about symptoms (e.g. diarrhea nausea, fatigue), quality of life, and functional status. All questions except rash were rated on a 1-5 scale, with higher scores indicating more adverse values. Rash was graded as a binary variable (present/absent). Adherence to in-clinic PROs in our clinic has been shown to be 71.2%.²⁷

Outcome

The primary outcome was 180-day mortality from the date of the index encounter at an oncology practice. We chose 180-day mortality because it is a common indicator of short-term mortality and is often used as a criterion for hospice referral.¹⁹ Date of death was derived from the first date of death recorded in either the EHR (Clarity database) or the Social Security Administration (SSA) Death Master File, matched to UPHS patients by social security number and date of birth. The SSA Death Master File contains information on the death of anyone holding a Social Security Number as reported by relatives of deceased individuals, funeral directors, financial institutions, and postal authorities.³⁰

EHR Algorithm

To develop an algorithm based on EHR variables alone, we applied the least absolute shrinkage and selection operator (LASSO) logistic regression model for variable selection and model building.³¹ For this model, we used the 559 EHR variables described in the EHR features section above as covariates and observed mortality status as outcome. All patients with EHR features were included in the algorithm. The odds ratio coefficient estimates were penalized toward zero to deal with overfitting, with the tuning parameter for controlling the degree of shrinkage determined using 10-fold cross-validation. We labeled this LASSO algorithm as the EHR algorithm.

PRO Algorithm

To develop an algorithm based on PROs alone, we fitted a logistic regression where all of the 12 PROs are included as covariates, with observed 180-day mortality as outcome. Only patients with PRO data were included in the model. We labeled this regression as the PRO algorithm.

EHR+PRO Algorithm

To develop an algorithm that includes both EHR and PRO variables, we applied a data augmentation method to fit the prediction algorithm and estimate the AUC and AUPRC that makes full use of all available EHR (N=8,600) and PRO (m=4692) data.³²⁻³⁴ We chose this method because it accounts for the monotone missingness structure where PRO data is fully available for a subset of patients and EHR data is available for all. In this method, first the predicted probabilities were generated from the EHR algorithm built using the adaptive LASSO method for all patients. Then we fit a logistic regression algorithm with a novel offset term that includes the logit of these probabilities together with all 12 PRO variables as predictors on the subset of patients who have PRO data, and the resultant algorithm is our EHR+PRO algorithm. Built upon a widely used statistical method for analyzing data with a monotone missingness structure^{32, 34}, our method adjusts for potential non-representativeness of the PRO subset and makes use of the EHR data beyond that in the PRO subset to achieve improved statistical efficiency for estimating AUC and AUPRC. Through utilizing the fitted probabilities in the final model, our approach effectively accommodates the high dimensionality of the EHR data. We labeled this regression as the EHR+PRO algorithm.

Statistical Analysis

We used descriptive statistics to compare the characteristics of the study population, stratified by whether PROs were collected. Standard mean differences and P-values were reported indicating whether the distribution was balanced or not across the stratification.

We first explored associations between (1) PRO features and mortality, and (2) PRO features and 180-day mortality risk as predicted from the EHR only model, in order to identify which PRO features were most associated with the outcome and to inform to what extent specific PROs may augment ML performance. We fit separate logistic regression models with 180-day mortality and 180-day mortality risk as the outcomes and each PRO as the only covariate. To further investigate the independent association between PROs and mortality, we also fit a two-variable logistic regression model that measured the association between each PRO and mortality, adjusted for the continuous 180-day mortality risk predicted from the EHR algorithm.

Finally, the performance of the 3 different algorithms (EHR, PRO, and EHR+PRO) were assessed by calculating area under the receiver operator characteristic curve (AUC), which was our primary performance metric. The true positive rate (TPR) at a previously specified 10% risk threshold³⁶ was our secondary performance metric. To further describe predictive performance, we calculated area under the precision-recall curve (AUPRC), which may be a better indicator of discrimination for rare outcomes³⁵, and false positive rates (FPRs). In addition, we examined heterogeneity of performance of the two-phase algorithm by training separate EHR+PRO algorithms in subgroups defined by cancer type, stage, and patient demographics. 95% confidence Intervals for each performance metric were derived using bootstrapping, where the data was resampled 1000 times. Bootstrapped confidence intervals were used to assess for statistically significant performance differences among the EHR, PRO, and EHR+PRO algorithms. To describe algorithm calibration, we calculated calibration slopes for each of the 3 algorithms for high risk individuals; a perfectly calibrated algorithm would have a calibration slope of 1.³⁷

All analyses were conducted using R version 3.6.0.

Subgroup Analyses

In order to assess for heterogeneity in the relationship between the three algorithms, we performed subgroup analyses comparing EHR+PRO vs. EHR performance, stratified by patient- and cancer-specific factors. We split the data into subgroups based on cancer type and patient demographics, stage, and calculated performance measures by training separate algorithms in each subgroup. We used the original algorithm fit on the full data to obtain model coefficients, and again used 1000 bootstrap samples from each subgroup to calculate each accuracy measure and obtain confidence intervals.

Results

Cohort Demographics

The study cohort consisted of 8,600 patients who had 40,955 encounters (median encounters per patient 3, interquartile range [IQR] 2,6) during the study period. The median age was 64.4 years [IQR 55.1-72.2], 4438 (51.6%) were female, 6336 (73.7%) were non-Hispanic White, 1489 (17.3%) were non-Hispanic Black, 144 (1.7%) were Hispanic, and 4419 (51.4%) had Medicare insurance. The median number of comorbidities was 3 [IQR 2-4]. The most common malignancies included in the cohort were lymphoma (14.7%), gastrointestinal (13.1%), breast (12.1%), and thoracic (10.8%).

Study Population Characteristics

Of 8600 patients in the cohorts, 485 (5.6%) died during the 180-day follow-up period. 4692 (54.5%) patients had completed all 12 PRO assessments during the study period. Compared to patients who did not complete PRO assessments, patients who completed PRO assessments were more likely to be younger [mean age 63.4 years vs 65.4 years; P<0.001]; White [3596 (76.6%) vs. 2710 (70.1%); P<0.001]; and have managed care insurance [1587 (33.8%) vs. 1191 (28.2%)].

PRO Associations with Observed Mortality and EHR Mortality Risk

In unadjusted analyses, adverse PROs were associated with higher observed 180-day mortality for all PROs except for numbness & tingling and rash. Worse patient-reported performance status (odds ratio [OR], 2.0; 95% confidence interval [CI], 1.80-2.23), quality of life (OR, 1.91; 95% CI, 1.70-2.15), decreased appetite (OR, 1.83; 95% CI, 1.64-2.03), and fatigue (OR, 1.74; 1.58-1.93) had the strongest association with observed mortality (FIGS. 1B and 1C). After adjusting for EHR mortality risk, associations between adverse PROs and observed mortality remained significant for the following PROs: performance status, quality of life, fatigue, shortness of breath, anxiety, sadness, constipation, decreased appetite, and nausea (range of adjusted ORs 1.12-1.38). Adverse PROs were also associated with higher EHR mortality risk for all PROs except for rash. Associations between PRO scores and EHR mortality risk were strongest for patient-reported performance status (mean score in 1st vs. 4th quartile of EHR mortality risk 1.5 vs. 2.1, p=0.04), quality of life (2.2 vs. 2.9, p=0.03), and fatigue (2.0 vs. 2.7, p=0.04).

Algorithm Performance

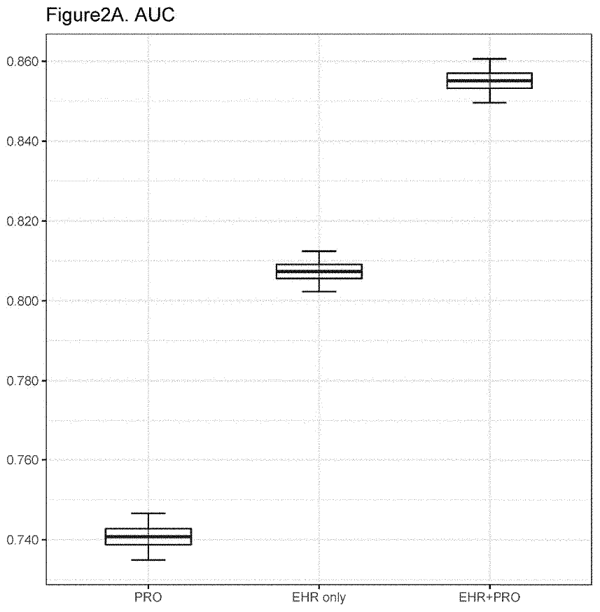

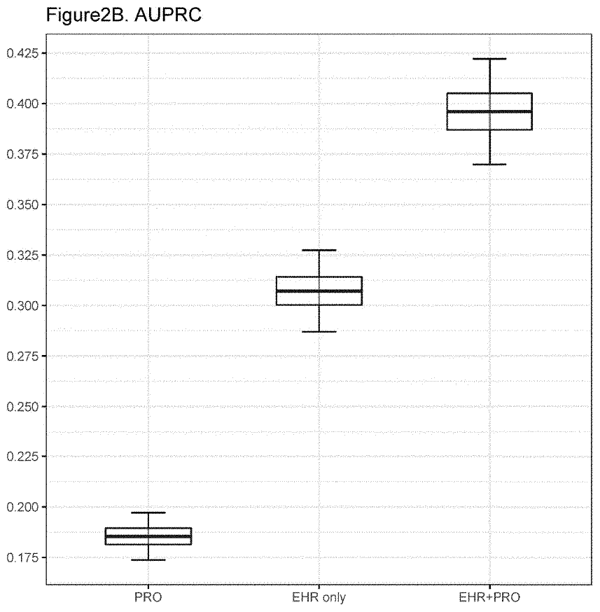

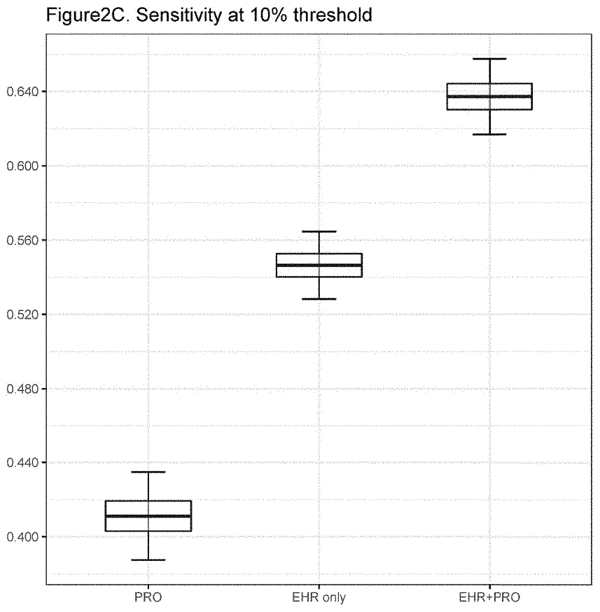

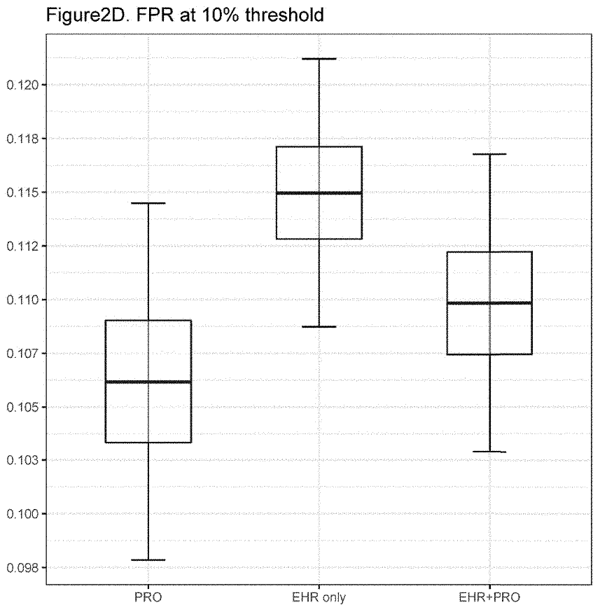

AUC and TPR were all greater for the EHR+PRO algorithm than for the EHR or PRO algorithms alone (FIGS. 2A-2D). The AUC of the EHR +PRO algorithm (0.86; 95% CI: 0.85-0.86) was significantly higher than that of the EHR (0.81; 95% CI: 0.80-0.81, p<0.001) and PRO (0.74; 95% CI: 0.74-0.74, p<0.001) algorithms (FIG. 2A). The AUPRC of the EHR+PRO algorithm (0.40; 95% CI: 0.37-0.42) was significantly higher than that of the EHR (0.31; 95% CI: 0.29-0.33) and PRO (0.19; 95% CI:0.17-0.20) algorithms (FIG. 2B). The TPR of the EHR+PRO algorithm (0.64; 95% CI: 0.62-0.66) was significantly higher than that of the EHR (0.55; 95% CI:0.53-0.56) and PRO (0.41; 95% CI: 0.39-0.43) algorithms (FIG. 2C). There was no difference in false positive rates among the EHR+PRO (0.11; 95% CI: 0.10-0.12), EHR (0.11; 95% CI: 0.11-0.12) and PRO (0.11; 95% CI: 0.10-0.11) algorithms (FIG. 2D). There was no significant difference in calibration slopes among the EHR+PRO (0.60; 95% CI: 0.41-0.80), EHR (0.71; 95% CI: 0.54-0.89) and PRO (0.65; 95% CI: 0.25-0.75) algorithms.

Subgroup Analyses

Overall performance of the EHR+PRO algorithm was similar across cancer types and cancer stage, with the exception of primary CNS malignancies, for which AUC, AUPRC, and TPR were significantly lower than other primary sites. Compared to the EHR alone algorithm, the EHR+PRO algorithm had higher AUC across all cancer site, race, age and age subgroups, with the exception of patients with stage IV malignancies, where performance was similar (AUC 0.85 [0.83-0.87] vs. 0.82 [0.80-0.84]).

Discussion

In a cohort of patients with cancer treated at a large tertiary cancer center, ML algorithms based on structured EHR and PRO data outperformed algorithms based on EHR or PRO data alone in predicting short-term mortality. Adverse PROs had strong associations with 180-day mortality, particularly for patient-reported functional status, quality of life, and fatigue. Moreover, performance of the EHR+PRO algorithm was consistently better than an algorithm based on EHR data alone across important cancer-specific and demographic subgroups. Collectively, these findings suggest that routinely-collected patient-reported symptoms, quality of life, and performance status have considerable independent prognostic value over and above structured EHR data and augment ML models based on EHR data alone. Future predictive algorithms should prioritize incorporation of patient-reported data in addition to structured administrative data.

Prognostic modeling and risk-stratification represent a novel use case for use of PROs in routine clinical care. Primary use cases for PROs are in clinical symptom management and toxicity monitoring during clinical trials.⁷ Randomized trials have shown that routine web-based collection of PROs among patients with advanced cancers is associated with improvements in patient-reported quality of life, satisfaction, patient-physician communication, and overall survival.^{3, 6, 38} As a result, many PROs are used as part of outcome or process metrics for evaluating the quality of oncology care, and routine PRO collection is recommended by consensus guidelines.³⁹ However, use of routinely-collected PROs as part of risk stratification, including prognostic risk stratification, is rare in practice.

Our results suggest that PROs have a key role to play in mortality risk prediction and stratification in oncology, even in the era of more advanced ML methods. While prior retrospective studies have found that adverse quality of life and symptoms such as depression, fatigue, and pain are independently associated with poorer survival^{24, 40, 41} few studies have demonstrated the independent prognostic value of PROs in contemporary machine learning algorithms, which are primarily based on laboratory, demographic, and comorbidity data. Indeed, our study suggests that PROs are only modestly correlated with EHR-predicted mortality risk, and there is likely additional independent prognostic value of PROs that would be of benefit in ML algorithms. In one ML algorithm to predict survival, investigators derived patient-reported symptoms from clinical notes using natural language processing.²¹ However, clinical notes may not adequately capture actual patient symptom burden and quality of life and may be subject to clinician biases in reporting symptoms; indeed, there is significant discordance between actual patient-reported symptoms and their documentation in the EHR.⁴² This is a key limitation of NLP to extract patient-reported symptoms and quality of life. Relying on routinely-collected patient-reported outcomes is likely a better way to capture symptoms in order to maximally improve performance of predictive algorithms.

Integration of PROs into risk stratification could address gaps in supportive care delivery among patients with cancer. We have previously shown that ML algorithms based on the same structured EHR features used in this study accurately predict 180-day mortality among patients with cancer.^{18, 25} In a recent prospective trial, an ML algorithm linked to automated alerts to clinicians quadrupled rates of ACP discussions among patients with cancer and other advanced illnesses.³⁶ Similar studies have suggested that automated ML prognostic algorithms may successfully trigger palliative care consultation.⁴³ A key barrier to implementing such prognostic algorithms may be under-identification of high-risk patients, as true positive rates are generally below 50% in such algorithms. However, such algorithms did not prioritize integration of routine PROs as part of these ML algorithms. The predictive performance of the EHR+PRO algorithm exceeded that of the EHR algorithm alone, and the TPR (64%) of our EHR+PRO algorithm at a 10% threshold generally outperformed TPRs seen in other published algorithms.^{19, 20}

Our findings establish the role of patient-reported outcomes as predictors in mortality prediction algorithms and suggest that routinely-collected PROs may augment commonly used risk stratification models in oncology. Prior attempts at integrating PROs into prognostic algorithms have relied primarily on complete case analyses, which may not utilize representative populations. Our method does not require complete presence of PRO data for predictive algorithm development. This is key for real-world practice, where not all patients will be adherent to PRO collection.

Accurate identification of patients at risk of short-term mortality is important in oncology given guidelines around early palliative care and advance care planning for high-risk patients with cancer.^{44, 45} We have previously demonstrated that algorithm-based “nudges” dramatically increase use of such guideline-based care in oncology.³⁶ Accuracy of such models may be a key barrier to integration in clinical practice. Incorporating PROs in such tools could improve accuracy and aid clinicians' risk assessments for patients with cancer as well as serving as a point-of-care prompt to consider discussions about goals and end-of-life preferences. These algorithms are flexible and can account for increasing availability of structured genetic and molecular information, which will likely increase and further improve model performance.

In conclusion, among 8600 patients with cancer seen at a tertiary medical oncology practice, a ML algorithm that integrated 12 routinely-collected patient reported outcomes about symptoms, quality of life, and performance status with over 500 electronic health record features to predict 180-day mortality improved AUC by 0.05-0.12 and TPR by 9-23 percentage-points, compared to algorithms based on electronic health record or patient-reported outcome data alone. The EHR+PRO algorithm improved performance across all relevant cancer-specific and demographic subgroups. Additionally, several PROs—notably performance status, quality of life, decreased appetite, and fatigue—had the strongest independent associations with mortality. Our findings suggest that PROs can significantly improve performance of predictive algorithms in oncology and that flexible algorithms that utilize PROs when they are available should be prioritized.

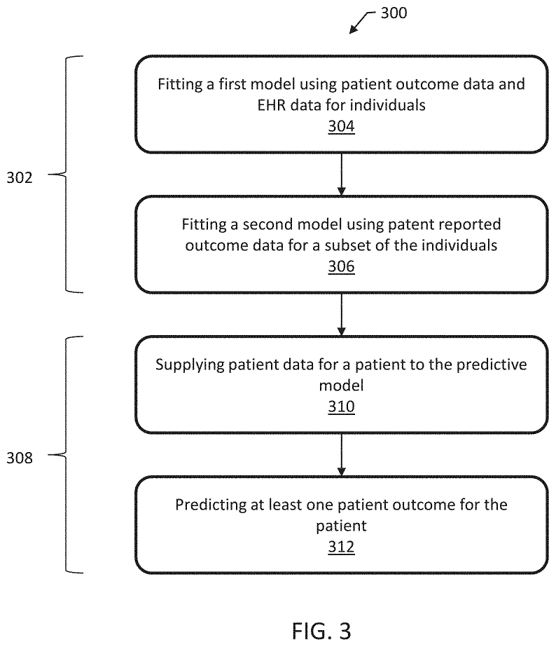

FIG. 3 is a flow diagram of an example method 300 for predicting patient outcomes. The method 300 is performed by a system of one or more computers.

The method 300 includes a training phase 302 for training a predictive model. The training phase 302 includes fitting a first model using patient outcome data for a number of individuals and HER data for the individuals (304). The training phase 302 also includes fitting a second model using patient reported outcome data for a subset of the individuals (306).

The method 300 includes a production phase 308 for predicting patient outcomes using the predictive model. The production phase 308 includes supplying patient data for a patient to the predictive model (310). The production phase 308 includes using the predictive model to predict at least one patient outcome for the patient (312). The patient outcome can be outputted, for example, by displaying the patient outcome on a display screen to a caregiver, or by sending the patient outcome to a remote computer system.

In some examples, training the predictive model includes generating predicted probabilities from the electronic health records. Training the predictive model can include obtaining a summary score for a plurality of electronic health record covariates.

In some examples, fitting the first model includes applying the least absolute shrinkage and selection operator (LASSO) logistic regression model. Fitting the second model can include fitting a logistic regression model with an offset term.

In some examples, the electronic health record data includes one or more of: demographic variables, comorbidities, and laboratory data. Patient reported outcome data can include patient response to questions about one or more of: symptoms, quality of life, and functional status. The patient outcome data can include mortality data, and using the predictive model to predict at least one patient outcome can then include predicting mortality for the patient.

Accordingly, while the methods, systems, and computer readable media have been described herein in reference to specific embodiments, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein.

Various combinations and sub-combinations of the structures and features described herein are contemplated and will be apparent to a skilled person having knowledge of this disclosure. Any of the various features and elements as disclosed herein may be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

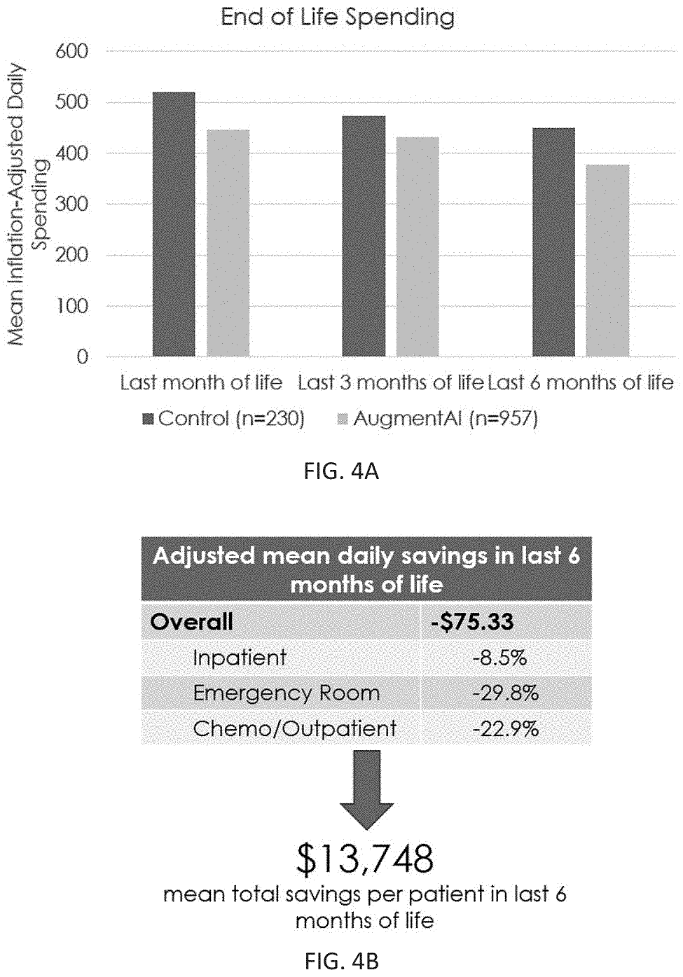

FIGS. 4A-4B are charts illustrating example data for the usefulness of an example system that leads to cost savings in end of life spending. FIG. 4A is a chart illustrating, in an example study using the system, spending for a control group compared to spending for a group using the system. FIG. 4B chart showing example adjusted mean daily savings in last six months of life from the example study.

### REFERENCES

1. Bubis L D, Davis L, Mahar A, et al: Symptom Burden in the First Year After Cancer Diagnosis: An Analysis of Patient-Reported Outcomes. J Clin Oncol Off J Am Soc Clin Oncol 36:1103-1111, 2018
2. Lage D E, E I-Jawahri A, Fuh C-X, et al: Functional Impairment, Symptom Burden, and Clinical Outcomes Among Hospitalized Patients With Advanced Cancer. J Natl Compr Canc Netw 18:747-754, 2020
3. Basch E, Deal A M, Kris M G, et al: Symptom Monitoring With Patient-Reported Outcomes During Routine Cancer Treatment: A Randomized Controlled Trial. J Clin Oncol 34:557-565, 2016
4. Basch E, Barbera L, Kerrigan C L, et al: Implementation of Patient-Reported Outcomes in Routine Medical Care. Am Soc Clin Oncol Educ Book 122-134, 2018
5. Yang L Y, Manhas D S, Howard A F, et al: Patient-reported outcome use in oncology: a systematic review of the impact on patient-clinician communication. Support Care Cancer Off J Multinatl Assoc Support Care Cancer 26:41-60, 2018
6. Basch E, Deal A M, Dueck A C, et al: Overall Survival Results of a Trial Assessing Patient-Reported Outcomes for Symptom Monitoring During Routine Cancer Treatment. JAMA 318:197-198, 2017
7. Basch E, Barbera L, Kerrigan C L, et al: Implementation of Patient-Reported Outcomes in Routine Medical Care. Am Soc Clin Oncol Educ Book 122-134, 2018
8. Krishnan M, Temel J S, Wright A A, et al: Predicting life expectancy in patients with advanced incurable cancer: a review. J Support Oncol 11:68-74, 2013
9. Chow E, Harth T, Hruby G, et al: How accurate are physicians' clinical predictions of survival and the available prognostic tools in estimating survival times in terminally ill cancer patients? A systematic review. Clin Oncol R Coll Radiol G B 13:209-218, 2001
10. White N, Reid F, Harris A, et al: A Systematic Review of Predictions of Survival in Palliative Care: How Accurate Are Clinicians and Who Are the Experts? PloS One 11:e0161407, 2016
11. Christakis N A, Lamont E B: Extent and determinants of error in doctors' prognoses in terminally ill patients: prospective cohort study. BMJ 320:469-472, 2000
12. Tang S T, Chen C H, Wen F-H, et al: Accurate Prognostic Awareness Facilitates, Whereas Better Quality of Life and More Anxiety Symptoms Hinder End-of-Life Care Discussions: A Longitudinal Survey Study in Terminally III Cancer Patients' Last Six Months of Life. J Pain Symptom Manage 55:1068-1076, 2018
13. Nipp R D, Greer J A, E I-Jawahri A, et al: Coping and Prognostic Awareness in Patients With Advanced Cancer. J Clin Oncol Off J Am Soc Clin Oncol 35:2551-2557, 2017
14. Lundquist G, Rasmussen B H, Axelsson B: Information of imminent death or not: does it make a difference? J Clin Oncol Off J Am Soc Clin Oncol 29:3927-3931, 2011
15. Zhang B, Wright A A, Huskamp H A, et al: Health care costs in the last week of life: associations with end-of-life conversations. Arch Intern Med 169:480-488, 2009
16. E I-Jawahri A, Traeger L, Park E R, et al: Associations among prognostic understanding, quality of life, and mood in patients with advanced cancer. Cancer 120:278-285, 2014
17. Finlay E, Casarett D: Making difficult discussions easier: using prognosis to facilitate transitions to hospice. C A Cancer J Clin 59:250-263, 2009
18. Parikh R B, Manz C, Chivers C, et al: Machine Learning Approaches to Predict 6-Month Mortality Among Patients With Cancer. JAMA Netw Open 2:e1915997, 2019
19. Elfiky A A, Pany M J, Parikh R B, et al: Development and Application of a Machine Learning Approach to Assess Short-term Mortality Risk Among Patients With Cancer Starting Chemotherapy. JAMA Netw Open 1:e180926—e180926, 2018
20. Bertsimas D, Dunn J, Pawlowski C, et al: Applied Informatics Decision Support Tool for Mortality Predictions in Patients With Cancer [Internet]. JCO Clin Cancer Inform, 2018[cited 2019 Jan. 11] Available from: http://ascopubs.org/doi/abs/10.1200/CCI.18.00003
21. Gensheimer M F, Henry A S, Wood D J, et al: Automated Survival Prediction in Metastatic Cancer Patients Using High-Dimensional Electronic Medical Record Data. J Natl Cancer Inst 111:568-574, 2019
22. Gensheimer M F, Aggarwal S, Benson K R K, et al: Automated model versus treating physician for predicting survival time of patients with metastatic cancer. J Am Med Inform Assoc JAMIA, 2020
23. Baum L V M, Friedman D: The Uncertain Science of Predicting Death. JAMA Netw Open 3:e201736, 2020
24. Stukenborg G J, Blackhall L J, Harrison J H, et al: Longitudinal patterns of cancer patient reported outcomes in end of life care predict survival. Support Care Cancer 24:2217-2224, 2016
25. Manz C R, Chen J, Liu M, et al: Validation of a Machine Learning Algorithm to Predict 180-Day Mortality for Outpatients With Cancer. JAMA Oncol, 2020
26. Collins G S, Reitsma J B, Altman D G, et al: Transparent Reporting of a multivariable prediction model for Individual Prognosis Or Diagnosis (TRIPOD): The TRIPOD Statement. Ann Intern Med 162:55, 2015
27. Elixhauser A, Steiner C, Harris D R, et al: Comorbidity measures for use with administrative data. Med Care 36:8-27, 1998
28. National Cancer Institute Division of Cancer Control and Population Sciences: Patient-Reported Outcomes version of the Common Terminology Criteria for Adverse Events (PRO-CTCAE) [Internet][cited 2020 Oct. 6] Available from: https://healthcaredelivery.cancer.gov/pro-ctcae/29.
29. Hays R D, Bjorner J B, Revicki D A, et al: Development of physical and mental health summary scores from the patient-reported outcomes measurement information system (PROMIS) global items. Qual Life Res 18:873-880, 2009
30. NTIS: Limited Access Death Master File Download [Internet][cited 2019 Aug. 28] Available from: https://dmf.ntis.gov/31.
31. Friedman J, Hastie T, Tibshirani R, et al: glmnet: Lasso and Elastic-Net Regularized Generalized Linear Models [Internet]. 2021[cited 2021 Apr. 28] Available from: https://CRAN.R-project. org/package=glmnet
32. BRESLOW N E, CAIN K C: Logistic regression for two-stage case-control data. Biometrika 75:11-20, 1988
33. Cain K C, Breslow N E: Logistic regression analysis and efficient design for two-stage studies. Am J Epidemiol 128:1198-1206, 1988
34. Scott A J, Wild C J: Fitting Logistic Models Under Case-Control or Choice Based Sampling. J R Stat Soc Ser B Methodol 48:170-182, 1986
35. Boyd K, Eng K H, Page C D: Area under the Precision-Recall Curve: Point Estimates and Confidence Intervals, in Blockeel H, Kersting K, Nijssen S, et al (eds): Machine Learning and Knowledge Discovery in Databases. Springer Berlin Heidelberg, 2013, pp 451-466
36. Manz C R, Parikh R B, Small D S, et al: Effect of Integrating Machine Learning Mortality Estimates With Behavioral Nudges to Clinicians on Serious Illness Conversations Among Patients With Cancer: A Stepped-Wedge Cluster Randomized Clinical Trial. JAMA Oncol e204759, 2020
37. Stevens R J, Poppe K K: Validation of clinical prediction models: what does the “calibration slope” really measure? J Clin Epidemiol 118:93-99, 2020
38. Velikova G, Booth L, Smith A B, et al: Measuring Quality of Life in Routine Oncology Practice Improves Communication and Patient Well-Being: A Randomized Controlled Trial. J Clin Oncol 22:714-724, 2004
39. Basch E, Snyder C, McNiff K, et al: Patient-Reported Outcome Performance Measures in Oncology. J Oncol Pract 10:209-211, 2014
40. Saint-Maurice P F, Troiano R P, Bassett D R, et al: Association of Daily Step Count and Step Intensity With Mortality Among U S Adults. JAMA 323:1151-1160, 2020
41. van Seventer E E, Fish M G, Fosbenner K, et al: Associations of baseline patient-reported outcomes with treatment outcomes in advanced gastrointestinal cancer. Cancer, 2020
42. Pakhomov S V, Jacobsen S J, Chute C G, et al: Agreement between patient-reported symptoms and their documentation in the medical record. Am J Manag Care 14:530-539, 2008
43. Courtright K R, Chivers C, Becker M, et al: Electronic Health Record Mortality Prediction Model for Targeted Palliative Care Among Hospitalized Medical Patients: a Pilot Quasi-experimental Study. J Gen Intern Med 34:1841-1847, 2019
44. Ferrell B R, Temel J S, Temin S, et al: Integration of Palliative Care Into Standard Oncology Care: American Society of Clinical Oncology Clinical Practice Guideline Update. J Clin Oncol 35:96-112, 2016
45. Temel J S, Greer J A, E I-Jawahri A, et al: Effects of Early Integrated Palliative Care in Patients With Lung and GI Cancer: A Randomized Clinical Trial. J Clin Oncol 35:834-841, 2017

What is claimed is:

1. A method for predicting patient outcomes, the method comprising:
training, using at least one processor, a predictive model by:
fitting a first model using patient outcome data for a plurality of individuals and electronic health record data for the plurality of individuals; and
fitting a second model using patient reported outcome data for a subset of the plurality of individuals;
supplying patient data for a patient to the predictive model; and
using the predictive model to predict at least one patient outcome for the patient.

2. The method of claim 1, wherein training the predictive model comprises generating a plurality of predicted probabilities from the electronic health records.

3. The method of claim 1, wherein training the predictive model comprises obtaining a summary score for a plurality of electronic health record covariates.

4. The method of claim 1, wherein fitting the first model comprises applying the least absolute shrinkage and selection operator (LASSO) logistic regression model.

5. The method of claim 1, wherein fitting the second model comprises fitting a logistic regression model with an offset term.

6. The method of claim 1, wherein the electronic health record data includes one or more of: demographic variables, comorbidities, and laboratory data.

7. The method of claim 1, wherein patient reported outcome data includes patient response to questions about one or more of: symptoms, quality of life, and functional status.

8. The method of claim 1, wherein the patient outcome data comprises mortality data, and wherein using the predictive model to predict at least one patient outcome comprises predicting mortality for the patient.

9. A system comprising:
at least one processor and memory; and
a predictive trainer implemented using the at least one processor and configured for training a predictive model by:
fitting a first model using patient outcome data for a plurality of individuals and electronic health record data for the plurality of individuals; and
fitting a second model using patient reported outcome data for a subset of the plurality of individuals;
a predictor implemented using the at least one processor and configured for supplying patient data for a patient to the predictive model and using the predictive model to predict at least one patient outcome for the patient.

10. The system of claim 9, wherein training the predictive model comprises generating a plurality of predicted probabilities from the electronic health records.

11. The system of claim 9, wherein training the predictive model comprises obtaining a summary score for a plurality of electronic health record covariates.

12. The system of claim 9, wherein fitting the first model comprises applying the least absolute shrinkage and selection operator (LASSO) logistic regression model.

13. The system of claim 9, wherein fitting the second model comprises fitting a logistic regression model with an offset term.

14. The system of claim 9, wherein the electronic health record data includes one or more of: demographic variables, comorbidities, and laboratory data.

15. The system of claim 9, wherein patient reported outcome data includes patient response to questions about one or more of: symptoms, quality of life, and functional status.

16. The system of claim 9, wherein the patient outcome data comprises mortality data, and wherein using the predictive model to predict at least one patient outcome comprises predicting mortality for the patient.

17. A non-transitory computer readable medium storing executable instructions that when executed by at least one processor of a computer control the computer to perform operations comprising:

18. The non-transitory computer readable medium of claim 17, wherein training the predictive model comprises generating a plurality of predicted probabilities from the electronic health records.

19. The non-transitory computer readable medium of claim 17, wherein training the predictive model comprises obtaining a summary score for a plurality of electronic health record covariates.

20. The non-transitory computer readable medium of claim 17, wherein fitting the first model comprises applying the least absolute shrinkage and selection operator (LASSO) logistic regression model.
